(19) United States
(12) Patent Application Publication
(10) Pub. No.: US 2023/0398156 A1
(43) Pub. Date: Dec. 14, 2023

(54) PHARMACEUTICAL COMPOSITION, FOR PREVENTING OR TREATING TENDON OR LIGAMENT DISEASES, COMPRISING UMBILICAL CORD-DERIVED STEM CELLS AS ACTIVE INGREDIENT

(71) Applicants: SEOUL NATIONAL UNIVERSITY R&DB FOUNDATION, Seoul (KR); ACESOSTEM BIOSTRATEGIES INC., Seoul (KR)

(72) Inventors: Hyun Chul JO, Seoul (KR); Ji-Hye YEA, Hwaseong-si (KR)

(73) Assignees: SEOUL NATIONAL UNIVERSITY R&DB FOUNDATION, Seoul (KR); ACESOSTEM BIOSTRATEGIES INC., Seoul (KR)

(21) Appl. No.: 18/034,110

(22) PCT Filed: May 18, 2021
(86) PCT No.: PCT/KR2021/006240

§ 371 Date: Apr. 27, 2023

(30) Foreign Application Priority Data
Oct. 27, 2020 (KR) 10-2020-0140504

(51) Int. Cl. A61K 35/51; A61P 19/04

(52) U.S. Cl. CPC A61K 35/51; A61P 19/04

(57) ABSTRACT

A composition having umbilical cord-derived stem cells as an active ingredient is disclosed. The composition containing umbilical cord-derived stem cells as an active ingredient, can prevent, relieve, or treat a tendon or ligament disease by regenerating and reconstructing a damaged tendon without side effects when applied for a tendon disease.

### TECHNICAL FIELD

The present disclosure relates to a composition having umbilical cord-derived stem cells as an active ingredient, particularly to a composition for preventing, relieving or treating a tendon or ligament disease by administering umbilical cord-derived stem cells alone and a use thereof.

### BACKGROUND ART

Tendon diseases that account for 45% of musculoskeletal injuries cause severe pain, activity disorders and economic burden. They are so severe as to affect 17 million Americans each year. Rest, physical therapy, exercise, surgical treatment, steroid injection, non-steroidal anti-inflammatory drugs, etc. have been used to treat tendon diseases. Such existing therapies are limited in that, e.g., symptoms recur afterwards because the underlying cause of the tendon diseases (degenerative changes of tendon tissues) cannot be resolved completely.

Since the supply of bloodstream to tendons or ligaments is relatively insufficient as compared to other tissues of the body and the tendon cells of damaged tendon does not participate in regeneration in most cases, it is difficult to completely recover the lost functions of tendons.

Therapeutic agents for the musculoskeletal system using mesenchymal stem cells are being developed recently to solve this problem. Mesenchymal stem cells (MSCs) are stem cells existing through the body including the ban marrow, which can differentiate into a variety of cell types, including fat cells, bone cells and cartilage cells. Thus, therapies using the stem cells have attracted many attentions and high efficiencies of stem cells in in-vitro experiments have been reported. However, when the stem cells are transplanted into animal models or humans, their efficiency is decreased remarkably. Stem cells exhibit quite different therapeutic effects depending on their types and diseases to be treated and also depending on the tissues from which they originate and culturing conditions. Especially, the therapeutic effects of therapeutic agents for other musculoskeletal system diseases are not achieved in many cases of tendon injury.

Despite these situations, the difference in the therapeutic effects of bone marrow-derived mesenchymal stem cells, adipose-derived mesenchymal stem cells, umbilical cord blood-derived mesenchymal stem cells and umbilical cord-derived mesenchymal stem cells on tendon injury has not been researched enough.

### DISCLOSURE

### Technical Problem

The inventors of the present disclosure made consistent efforts to discover substances capable of treating tendon or ligament diseases. They have researched the effect of recovering damaged tendon to a normal state while inhibiting side effects such as heterotopic ossification for the existing stem cells (bone marrow-derived mesenchymal stem cells, adipose-derived mesenchymal stem cells, umbilical cord blood-derived mesenchymal stem cells and umbilical cord-derived mesenchymal stem cells). As a result, they have identified that umbilical cord-derived mesenchymal stem cells regenerate and restore effectively without side effects by increasing the expression of tendon matrix genes and proteins, relieving tendon damage macroscopically without adhesion of the regenerated tendon with nearby tissues, preventing tendon degeneration histologically, restoring the arrangement of collagen fibers and inhibiting fibroblast deformation and heterotopic cartilage formation, and have completed the present disclosure.

The present disclosure is directed to providing a pharmaceutical composition for preventing or treating a tendon or ligament disease.

The present disclosure is also directed to providing a pharmaceutical composition for preventing or treating heterotopic ossification induced by a tendon or ligament disease.

Other problems to be solved and advantages of the present disclosure will become more apparent by the following detailed description, claims and drawings.

### Technical Solution

According to an aspect of the present disclosure, the present disclosure provides a pharmaceutical composition for preventing or treating a tendon or ligament disease, which contains umbilical cord-derived mesenchymal stem cells as an active ingredient.

The inventors of the present disclosure have made efforts to discover a new substance that regenerates and restores tendon or ligament tissues when tendon or ligament has been damaged. As a result, they have found out that umbilical cord-derived mesenchymal stem cells prevent, relieve or treat tendon or ligament diseases by regenerating and reconstructing damaged tendon without side effects.

In the present disclosure, “mesenchymal stem cells” refer to undifferentiated stem cells isolated from the tissues of human or mammals. The mesenchymal stem cells can be derived from various tissues, particularly from adipose tissue, bone marrow, umbilical cord, peripheral blood, placenta or umbilical cord blood. In a specific exemplary embodiment of the present disclosure, umbilical cord-derived mesenchymal stem cells are used. For isolation of the stem cells from each tissue, any technique known in the art may be used without special limitation.

It was identified that the umbilical cord-derived mesenchymal stem cells express the scleraxis gene, the type 1 collagen gene and the type 3 collagen gene at significantly higher levels than other stem cells (adipose-derived mesenchymal stem cells, umbilical cord blood-derived mesenchymal stem cells, bone marrow-derived mesenchymal stem cells, etc.) and have superior effect of regenerating and restoring damaged tendon.

Accordingly, a tendon or ligament disease can be prevented, relieved or treated easily with umbilical cord-derived mesenchymal stem cells only. But, it was identified that the effect of regenerating and restoring tendon is further improved as compared to the umbilical cord-derived mesenchymal stem cells if the Zkscan8 gene is overexpressed in the umbilical cord-derived mesenchymal stem cells. Specifically, the Zkscan8 gene may be transduced into the umbilical cord-derived mesenchymal stem cells.

The Zkscan8 gene may be represented by SEQ ID NO 1 or SEQ ID NO 3, and its protein may be represented by SEQ ID NO 2.

The umbilical cord-derived mesenchymal stem cells into which the Zkscan8 gene is transduced may be prepared by introducing a vector including the Zkscan8 gene.

In the present disclosure, the vector may be one or more selected from a group consisting of a linear DNA, a plasmid DNA and a recombinant viral vector, and the virus may be one or more selected from a group consisting of retrovirus, adenovirus, adeno-associated virus, herpes simplex virus and lentivirus.

The vector of the present disclosure may be delivered into a host cell by, for example, microinjection (Harland and Weintraub, J. Cell Biol. 101: 1094-1099 (1985)), calcium phosphate precipitation (Chen and Okayama, Mol. Cell. Biol. 7: 2745-2752 (1987)), electroporation (Tur-Kaspa et al., Mol. Cell Biol., 6: 716-718 (1986)), liposome-mediated transfection (Nicolau et al., Methods Enzymol., 149: 157-176 (1987)), DEAE-dextran method (Gopal, Mol. Cell Biol., 5: 1188-1190 (1985)) and gene bombardment (Yang et al., Proc. Natl. Acad. Sci., 87: 9568-9572 (1990)) although not being limited thereto.

In addition, the composition having the umbilical cord-derived mesenchymal stem cells as an active ingredient is characterized in that it exhibits a dual effect of regenerating or restoring tendon and inhibiting heterotopic ossification induced by a tendon disease. In general, when damaged tendon tissues are restored naturally or by therapeutic substances such as stem cells, side effects such as shoulder pain, retear and complications occur as heterotopic cartilage or ossification is induced. However, unlike other stem cells, the formation of heterotopic cartilage is inhibited and decreased remarkably for the composition according to the present disclosure (Test Example 6).

That is to say, the composition according to the present disclosure has superior effect of preventing, relieving or treating tendon or ligament diseases and, at the same time, inhibiting the side effect of heterotopic ossification. It is advantageous in that no additional drug or therapy for preventing side effects is necessary unlike the existing therapeutic agents for tendon diseases.

In the present disclosure, the ‘tendon disease’ may refer to gradual wearing of tendon caused by overuse or aging, chronic disorder or damage of tendon caused by tearing, tendon rupture, or separation of tendon from bone. Specifically, it may be one or more selected from a group consisting of Achilles tendon disease, patellar tendon disease, lateral epicondylitis, medial epicondylitis, plantar fasciitis, rotator cuff tendon disease, tenosynovitis, tendinopathy, tendinitis, tenosynovitis, tendon injury, tendon rupture and tendon avulsion.

The Achilles tendon disease, the patellar tendon disease or the rotator cuff tendon disease may be caused by the rupture of Achilles tendon, patellar tendon or rotator cuff tendon, inflammation of the tendon, degenerative change of collagen in the tendon due to overuse, damage of the tendon due to overuse or aging, and separation of the tendon from bone.

The tendon rupture is a disease caused by partial tearing of a tendon, which is a fibrous connective tissue that connects muscle to bone, or complete tearing into two pieces, and may be one or more selected from a group consisting of acute Achilles tendon rupture and patellar tendon rupture.

The tendinitis is a disease caused by the inflammation of a tendon caused by the microtear of the tendon occurring when abrupt and excessive load is applied to the musculotendinous unit, and may be one or more selected from a group consisting of Osgood-Schlatter disease, tenosynovitis, calcific tendinitis, patellar tendinitis, Achilles tendinitis, biceps tendinitis, rotator cuff tendinitis, lateral epicondylitis, supraspinatus tendinitis, triceps tendinitis and medial epicondylitis.

The tendinopathy is a tendon disease caused by chronic inflammation caused by the degenerative change of collagen of a tendon due to chronic overuse, and may be one or more selected from a group consisting of Achilles tendinopathy, patellar tendinopathy and bicipital tendinopathy.

The ligament disease may be one or more selected from a group consisting of cruciate ligament injury, ankle ligament injury, collateral ligament injury, ligament rupture and ligament sprain.

In the present specification, ‘prevention’ refers to the prevention of the onset of a disorder or a disease in a subject who has not been diagnosed to have the disorder or disease but has the possibility of having the disorder or disease.

In the present specification, ‘treatment’ refers to (a) prevention of a disorder, a disease or symptoms or the development thereof; (b) alleviation of a disorder, a disease or symptoms; or (c) removal of a disorder, a disease or symptoms. When the composition of the present disclosure is administered to a subject, it serves to prevent, remove or alleviate the development of the symptoms of a tendon or ligament disease by inducing the restoration of tendon tissues and the production of collagen. Accordingly, the composition of the present disclosure may be used as a composition for treating a tendon or ligament disease on its own or may be administered together with another pharmacological ingredient as a therapeutic adjuvant for the disease. Thus, in the present specification, the term ‘treatment’ or ‘therapeutic agent’ encompasses the meaning of ‘aid of treatment’ or ‘therapeutic adjuvant’.

In the present specification, ‘administration’ or ‘administer’ refers to the administration of a therapeutically effective amount of the composition of the present disclosure directly to a subject so that the same amount is formed in the body of the subject.

In the present disclosure, the ‘therapeutically effective amount’ refers to an amount of the composition of the present disclosure which is sufficient to provide a therapeutic or prophylactic effect in an individual to which the composition is administered and, thus, encompasses the meaning of ‘prophylactically effective amount’.

In the present specification, the ‘subject’ includes human, mouse, rat, guinea pig, dog, cat, horse, cow, pig, monkey, chimpanzee, baboon or rhesus monkey. Specifically, the subject of the present disclosure is human.

The pharmaceutical composition of the present disclosure may further contain a pharmaceutically acceptable carrier, and the carrier may be one commonly used in preparation, and includes lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, a sterilized aqueous solution, a nonaqueous solution, mineral oil, etc., although not being limited thereto. The pharmaceutical composition of the present disclosure may further contain a lubricant, a wetting agent, a sweetener, a flavorant, an emulsifier, a suspending agent, a preservative, etc. in addition to the above-described ingredients. Adequate pharmaceutically acceptable carriers and preparations are described in detail in Remington's Pharmaceutical Sciences (19th ed., 1995).

In addition, the pharmaceutical composition of the present disclosure may contain a carrier commonly used in the field of cell therapy because it contains the umbilical cord-derived mesenchymal stem cells or umbilical cord-derived mesenchymal stem cells in which the Zkscan8 gene is transduced as an active ingredient. The pharmaceutical composition of the present disclosure may be prepared into an injection for intra-tissue transplantation, an intravenous injection, a freeze-dried preparation for injection, etc. according to common methods. Specifically, it may be prepared into an injection for intra-tissue transplantation or an intravenous injection.

The pharmaceutical composition of the present disclosure may be administered orally or parenterally. Specifically, it may be administered parenterally, e.g., by intravenous injection, topical injection, intraperitoneal injection, etc.

The appropriate administration dosage of the pharmaceutical composition of the present disclosure varies depending on such factors as formulation method, mode of administration, the age, body weight, sex, pathological condition and diet of a patient, administration time, administration route, excretion rate and response sensitivity. An ordinarily skilled physician can easily determine and prescribe an administration dosage effective for the desired treatment or prevention. According to a specific exemplary embodiment of the present disclosure, the administration dosage of the pharmaceutical composition of the present disclosure may be 1 cell/kg or more, specifically 1 to 1×10¹⁰ cells/kg, more specifically 1×10³ to 1×10⁹ cells/kg, most specifically 1×10⁵ to 5×10⁸ cells/kg, per day.

The pharmaceutical composition of the present disclosure may be prepared as a single-dose or multiple-dose formulation using a pharmaceutically acceptable carrier and/or excipient according to a method that can be easily carried out by those having ordinary knowledge in the art to which the present disclosure belongs. The formulation may be a solution in an oily or aqueous medium, a suspension, an emulsion, an extract, a powder, a granule, a tablet or a capsule, and may further contain a dispersant or a stabilizer (Handbook of the Korean Pharmacopoeia, MoonSung Co., Korean Association of Pharmacy Education, 5th edition, p. 33-48, 1989). The pharmaceutical composition of the present disclosure may be used either alone or in combination with various existing therapeutic methods such as operation, surgery, medication, exercise therapy, physical therapy, rehabilitation therapy, radiation therapy, etc. When combination therapy is used, a tendon disease can be treated more effectively.

Accordingly, according to the present disclosure, umbilical cord-derived mesenchymal stem cells may be used as an active ingredient of a pharmaceutical composition for preventing or treating heterotopic ossification caused by a tendon or ligament disease.

Heterotopic ossification refers to formation of mature cartilage or bone in tissues where bone is not formed normally, and is distinguished from calcification in soft tissues.

The heterotopic ossification may be a complication caused by a tendon or ligament disease. Specifically, heterotopic cartilage or heterotopic bone may be formed around the tissues of a tendon or ligament.

The heterotopic ossification may be facilitated by stimulations such as burn, trauma, surgery or autotransplantation. The composition of the present disclosure, which contains umbilical cord-derived mesenchymal stem cells as an active ingredient, may prevent or treat heterotopic ossification induced by a tendon or ligament disease.

### Advantageous Effects

A composition according to the present disclosure, which contains umbilical cord-derived stem cells as an active ingredient, may prevent, relieve or treat a tendon or ligament disease by regenerating or reconstructing damaged tendon or ligament without side effects.

### BRIEF DESCRIPTION OF DRAWINGS

FIG. 1 shows a result of quantifying the expression level of the scleraxis gene in umbilical cord-derived mesenchymal stem cells (UC MSC) prepared in Example 1, adipose-derived mesenchymal stem cells (AD MSC) prepared in Comparative Example 1 and bone marrow-derived stem cells (BM MSC) prepared in Comparative Example 2 by RT-PCR.

FIG. 2 shows a result of quantifying the expression level of the type 1 collagen gene in umbilical cord-derived mesenchymal stem cells (UC MSC) prepared in Example 1, adipose-derived mesenchymal stem cells (AD MSC) prepared in Comparative Example 1 and bone marrow-derived stem cells (BM MSC) prepared in Comparative Example 2 by RT-PCR.

FIG. 3 shows a result of quantifying the expression level of the type 3 collagen gene in umbilical cord-derived mesenchymal stem cells (UC MSC) prepared in Example 1, adipose-derived mesenchymal stem cells (AD MSC) prepared in Comparative Example 1 and bone marrow-derived stem cells (BM MSC) prepared in Comparative Example 2 by RT-PCR.

FIGS. 4A and 4B show the images of the supraspinatus tendons of the supraspinatus-humerus complexes obtained at 2 and 4 weeks from control group (Saline), test group-UC (UC-MSC), comparison group-BM (BM-MSC) and comparison group-UCB (UCB-MSC) in Test Example 1. FIG. 4A shows the macroscopic images of the supraspinatus tendon of each group (tissues around the tendon were removed to clearly observe the defect of the tendon), and FIG. 4B shows the total macroscopic score of the supraspinatus tendon of each group.

FIG. 5 shows a result of recovering tendon tissues from the supraspinatus-humerus complexes obtained at 2 and 4 weeks from control group (Saline), test group-UC (UC-MSC), comparison group-BM (BM-MSC) and comparison group-UCB (UCB-MSC) in Test Example 1 and evaluating degenerative change and integration of structure. FIG. 5A shows the optical microscopic images of the tendons obtained at 2 and 4 weeks from control group (Saline), test group-UC (UC-MSC), comparison group-BM (BM-MSC) and comparison group-UCB (UCB-MSC) in Test Example 1 and stained with H&E (magnification: ×200). FIG. 5B shows the total degeneration scores of the tendons obtained at 2 and 4 weeks from control group (Saline), test group-UC (UC-MSC), comparison group-BM (BM-MSC) and comparison group-UCB (UCB-MSC) in Test Example 1, FIG. 5C-5I show the sub-parameters of the degeneration scores, and FIG. 5J shows the integration of structure.

FIG. 6 shows a result of evaluating collagen tissues and fibroblasts in tendon tissues of the supraspinatus-humerus complexes obtained at 2 and 4 weeks from control group (Saline), test group-UC (UC-MSC), comparison group-BM (BM-MSC) and comparison group-UCB (UCB-MSC) in Test Example 1. FIG. 6A shows the optical microscopic images of the tendons obtained at 2 and 4 weeks from control group (Saline), test group-UC (UC-MSC), comparison group-BM (BM-MSC) and comparison group-UCB (UCB-MSC) in Test Example 1 and stained with PSR (magnification: ×200). FIG. 6B shows a result of evaluating the collagen organization of the tendons obtained at 2 and 4 weeks from control group (Saline), test group-UC (UC-MSC), comparison group-BM (BM-MSC) and comparison group-UCB (UCB-MSC) in Test Example 1, and FIG. 6C shows a result of evaluating collage fiber coherence. FIG. 6D shows the images of fibroblasts in the tendons obtained at 2 and 4 weeks from control group (Saline), test group-UC (UC-MSC), comparison group-BM (BM-MSC) and comparison group-UCB (UCB-MSC) in Test Example 1 (magnification: ×400), FIG. 6E shows a result of evaluating fibroblast density, FIG. 6F shows a result of evaluating the nuclear aspect ratio of the fibroblasts, and FIG. 6G shows a result of evaluating nuclear orientation angle.

FIG. 7 shows a result of analyzing heterotopic change in the tendon tissues of the supraspinatus-humerus complexes obtained at 2 and 4 weeks from control group (Saline), test group-UC (UC-MSC), comparison group-BM (BM-MSC) and comparison group-UCB (UCB-MSC) in Test Example 1. FIG. 7A shows the images of the tendons obtained at 2 and 4 weeks from control group (Saline), test group-UC (UC-MSC), comparison group-BM (BM-MSC) and comparison group-UCB (UCB-MSC) in Test Example 1 and stained with Saf-O (magnification: ×200). FIG. 7B shows a result of measuring the glycosaminoglycan (GAG)-rich area of the tendons obtained at 2 and 4 weeks from control group (Saline), test group-UC (UC-MSC), comparison group-BM (BM-MSC) and comparison group-UCB (UCB-MSC) in Test Example 1, and FIG. 7C shows a result of measuring the area of ossification of the tendons obtained at 2 and 4 weeks from control group (Saline), test group-UC (UC-MSC), comparison group-BM (BM-MSC) and comparison group-UCB (UCB-MSC) in Test Example 1.

FIG. 8 shows a model for the structure of the ZSCAN family.

FIG. 9 shows the cleavage map of pscAAV-Zkscan 8.

FIG. 10 schematically shows the structure of a pscAAV-GFP vector and a pscAAV-Zkscan 8 vector.

### BEST MODE

Hereinafter, the present disclosure will be described in more detail through examples. The examples are provided only to describe the present disclosure in more detail and it will be obvious to those having ordinary knowledge in the art the scope of the present disclosure is not limited by the examples.

### EXAMPLES

The experiments of the present disclosure were approved by the Institutional Review Board and the Institutional Animal Care and Use Committee of Boramae Hospital and conducted according to approved procedures (IRB No. 16-2015-115 and IACUC_2019-0006).

### Example 1. Isolation and Subculturing of Umbilical Cord-Derived Mesenchymal Stem Cells

The tissues used in present disclosure were acquired under the consent of patients. Umbilical cords and tendon tissues were washed 2-3 times with calcium- and magnesium-free Dulbecco's phosphate-buffered saline supplemented with antibiotics (100 U/mL penicillin, 100 μg/mL streptomycin sulfate and 0.25 μg/mL amphotericin B (antibiotic-antimycotic solution; Welgene, Daegu, Korea)) to remove blood.

The umbilical cords obtained from patients who received Caesarean section were subjected to measurement of length and weight and then cut into a size of about 2-4 mm×2-4 mm using surgical scissors. Then, an amount corresponding to 1 g was inoculated onto a 150-cm² culture dish. After the umbilical cords were completely adhered to the culture dish, they were incubated in a culture medium (LG DMEM, 10% fetal bovine serum (FBS; Welgene, Daegu, Korea), antibiotic-antimycotic solution) at 37° C. while supplying 5% CO₂.

The cells were incubated at 37° C. for 2 hours in a high-glucose Dulbecco's modified Eagle medium (HG DMEM; Welgene, Daegu, Korea) supplemented with 0.3% type 2 collagenase (GIBCO) and antibiotics with light stirring. Then, after adding the same volume of a culture medium (HG DMEM, 10% FBS and antibiotic-antimycotic solution), undegraded cells were removed with a 100-μm cell filter. After centrifuging at 20° C. and 500 g for 15 minutes, the cells were collected and washed twice with a culture medium. After counting the isolated cells by trypan blue exclusion, they were transferred to a culture dish at a density of 2-5×10⁴ cells/cm² and incubated in a 5% CO₂ incubator at 37° C.

When the cells grew to fill about 60-80% of the culture dish, they were washed twice with DPBS and treated with 0.05% trypsin and 0.53 mM trypsin-EDTA (ethylenediamine tetraacetic acid) (Welgene, Daegu, Korea) for 3 minutes for isolation as single cells. The obtained umbilical cord-derived mesenchymal stem cells were counted by trypan blue exclusion and then subcultured by diluting with a culture medium to 1:4-1:6. Fresh cells subcultured for 3-5 passages were used for experiment.

### Example 2. Isolation and Subculturing of Umbilical Cord-Derived Mesenchymal Stem Cells

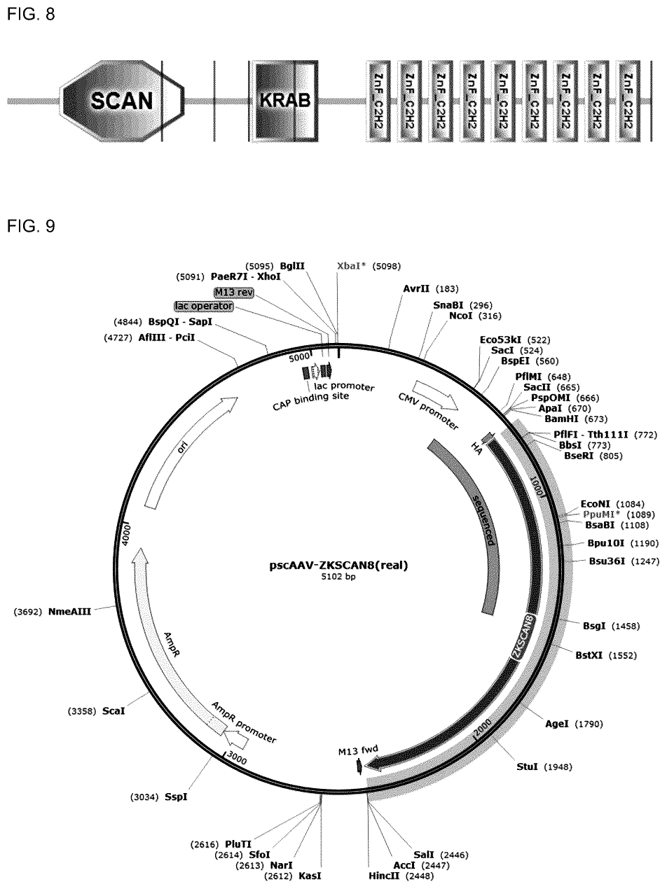

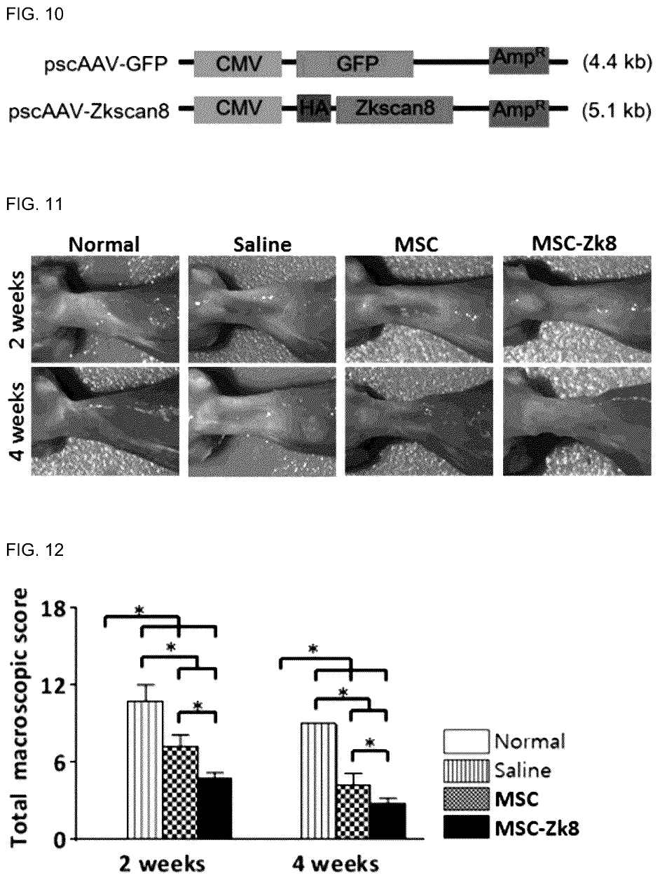

A pscAAV-GFP vector plasmid provided by Cell Biolabs (CA, USA) was used. After cleaving the GFP site with BamHI and SalI restriction enzymes, Zkscan8 (SEQ ID NO 1) was cloned into the site using primers (FP: 5′-AAGGATCCATGTACCCATACGATGTTCCAGATTACGCTATGGCGGAGGAAAGTC GG-3′, RP: 5′-AAGTCGACCTAGACTGAGATAGACTC-3′) and BamHI and SalI restriction enzymes. The cleavage map of the pscAAV-Zkscan 8 vector is shown in FIG. 8A, and the structures of the prepared pscAAV-GFP vector and pscAAV-Zkscan 8 vector are schematically shown in FIG. 8B. The sequence of the completed pscAAV-Zkscan8 was analyzed by sequencing. For production of viral packaging stocks, a total of three vectors (target expression vector, pAAV-RC and pHelper) were introduced into 293 cells. After collecting the culture medium including the cells 72 hours later, adenovirus including the Zkscan8 gene were acquired by repeating freezing and thawing. The prepared virus was used as a system for Zkscan 8 gene delivery system into the umbilical cord-derived mesenchymal stem cells prepared in Example 1.

### Comparative Example 1. Isolation and Subculturing of Adipose-Derived Mesenchymal Stem Cells

Adipose tissues were acquired under the consent of patients. In order to remove blood from the adipose tissues, they were washed 2-3 times with calcium- and magnesium-free Dulbecco's phosphate-buffered saline supplemented with antibiotics (100 U/mL penicillin, 100 μg/mL streptomycin sulfate, and 0.25 μg/mL amphotericin B (antibiotic-antimycotic solution; Welgene, Daegu, Korea)). The washed adipose tissues were sliced and treated with 0.1% type 1 collagenase (Sigma-Aldrich, St. Louis, MO, USA) for 60 minutes under the condition of 5% CO₂ and 37° C. with light stirring. Then, after adding the same volume of Dulbecco's phosphate-buffered saline (DPBS), the cells were recovered after conducting centrifugation at 20° C. and 1200 g for 10 minutes. After removing undegraded tissues using a 100-μm cell filter, the cells were washed twice with a culture medium (HG DMEM, 10% FBS and antibiotic-antimycotic solution). After counting the cells using a hemocytometer, the cells were inoculated onto a culture dish at a density of 1×10⁶ cells/cm² and incubated in a 5% CO₂ incubator at 37° C. for 24 hours. When the adipose-derived mesenchymal stem cells grew to fill about 60-80% of the culture dish, they were washed twice with DPBS and then treated with 0.05% trypsin and 0.53 mM trypsin-EDTA (ethylenediamine tetraacetic acid) (Welgene, Daegu, Korea) for 3 minutes for isolation as single cells. The obtained adipose-derived mesenchymal stem cells were counted by trypan blue exclusion and then subcultured after diluting with a culture medium to 1:4-1:6. Fresh cells subcultured for 3-5 passages were used for experiment.

### Comparative Example 2. Isolation and Subculturing of Bone Marrow-Derived Mesenchymal Stem Cells

Bone marrows were acquired under the consent of patients. The bone marrows were diluted with calcium (Ca²⁺)- and magnesium (Mg²⁺)-free Dulbecco's phosphate-buffered saline (DPBS, GIBCO, NY, USA) to 1:4. The diluted bone marrows were cautiously added to Ficoll-Paque™ Premium (GE Healthcare, Uppsala, Sweden) such that a surface layer could be formed to a final ration of 1:2. Then, after separating the layers by centrifuging at 20° C. and 400 g for 30 minutes, the uppermost supernatant was discarded and only the middle layer of monocytes was harvested. The harvasted monocyte was diluted with calcium- and magnesium-free Dulbecco's phosphate-buffered saline to 1:4 and then was centrifuged 400 g for 5 minutes at 20° C. to harvest cells. the cells were washed once again with calcium- and magnesium-free Dulbecco's phosphate-buffered saline. Then, after centrifuging at 20° C. and 400 g for 5 minutes, the supernatant was discarded and only the cells were remained. The collected cells were diluted with 10 mL of a low-glucose Dulbecco's modified Eagle's medium (DMEM) containing 10% inactivated FBS, 100 U/mL penicillin and 100 μg/mL streptomycin. A diluted cell solution was prepared by repeating centrifugation and addition of the medium twice. After measuring the number of cells in the solution using a hemocytometer, the cells were inoculated onto a culture dish at a density of 1×10⁵ cells/cm² and then incubated under the condition of 37° C. and 5% CO₂.

When the bone marrow-derived mesenchymal stem cells grew to fill about 60-80% of the culture dish, the cells were washed twice with DPBS and treated with 0.05% trypsin and 0.53 mM trypsin-EDTA (ethylenediamine tetraacetic acid) (Welgene, Daegu, Korea) for 3 minutes for isolation as single cells. The obtained bone marrow-derived mesenchymal stem cells were counted by trypan blue exclusion and then subcultured after diluting with a culture medium to 1:4-1:6. Fresh cells subcultured for 3-5 passages were used for experiment.

### Comparative Example 3. Isolation and Subculturing of Umbilical Cord Blood-Derived Mesenchymal Stem Cells

Umbilical cord blood-derived mesenchymal stem cells (HUXUB_01001, cyagne, 2255 martinmar, passage 3 purchased from Santa Clara, CA 95050, USA) were cultured using a special medium (HUXUB_90011). When the cells grew to fill about 60-80% of a culture dish, they were washed twice with DPBS and detached by treating with 0.05% trypsin and 0.53 mM trypsin-EDTA (ethylenediamine tetraacetic acid) (Welgene, Daegu, Korea) for 3 minutes. The cells were counted by trypan blue exclusion and then subcultured after diluting with a culture medium to 1:4-1:6. Fresh cells subcultured for 3-5 passages were used for experiment.

### TEST EXAMPLES

Statistical Analysis

All data were represented as mean±SD. The data were analyzed with one-way analysis of variance (ANOVA) with post hoc analysis using Bonferroni multiple comparison test. All statistical analyses were performed with SPSS software version 23 (IBM). Differences of P<0.050 were considered statistically significant.

### Test Example 1. Expression of Tendon-Specific Markers, Tendon Matrix Genes and Proteins

The expression of the scleraxis, type 1 collagen and type 3 collagen genes in the stem cells cultured for 3 passages or longer in Example 1, Comparative Example 1 and Comparative Example 2 was investigated(n=5).

1) Quantitative Reverse Transcription Polymerase Chain Reaction (qRT-PCR)

The expression of tendon-related genes (scleraxis, type 1 collagen and type 3 collagen genes) in the stem cells cultured for 3 passages or longer in Example 1, Comparative Example 1 and Comparative Example 2 was investigated.

First, after extracting total RNA using a HiYield Total RNA mini kit (Real Biotech Corporation, Taiwan), absorbance was measured at 260 nm and 280 nm using a spectrophotometer (NanoDrop, DE, USA) and the total RNA was quantified. Then, cDNA was synthesized from 1 μg of each total RNA using Superscript II reverse transcriptase (Invitrogen, CA. USA). The expression of the scleraxis, type 1 and type 3 collagen genes was monitored in real time by quantitative reverse transcription polymerase chain reaction (qRT-PCR) using Go Taq® probe qPCR and RT-qPCR systems (Promega, WI, USA), TaqMan® Gene Expression Assays (Applied Biosystems, Foster City, CA, USA) and LightCycler 480 (Roche Applied Science, Mannhein, Germany). The polymerase chain reaction was conducted by repeating 50 cycles of pre-denaturation at 95° C. for 10 minutes, denaturation at 95° C. for 15 seconds, annealing at 60° C. for 1 minute and extension at 72° C. for 4 seconds, followed by cooling at 40° C. for 30 seconds. Melting curve analysis was conducted by calculating 2-ΔCt, and the result of qRT-PCR was analyzed using the expression of GAPDH as a reference gene (Livak K J, Schmittgen T D. Analysis of relative gene expression data using real-time quantitative PCR and the 2-ΔΔCt method. Methods. 2001, 25: 402-408).

2) Result

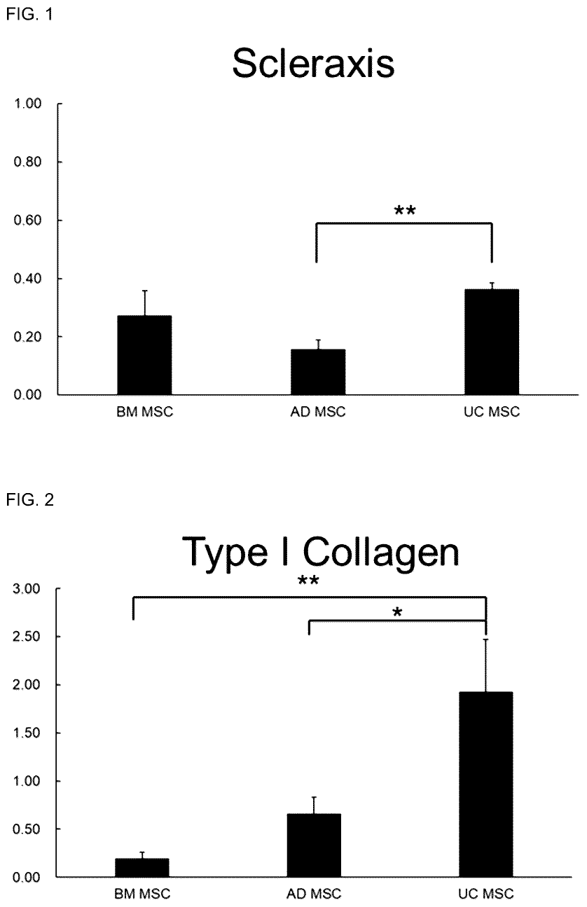

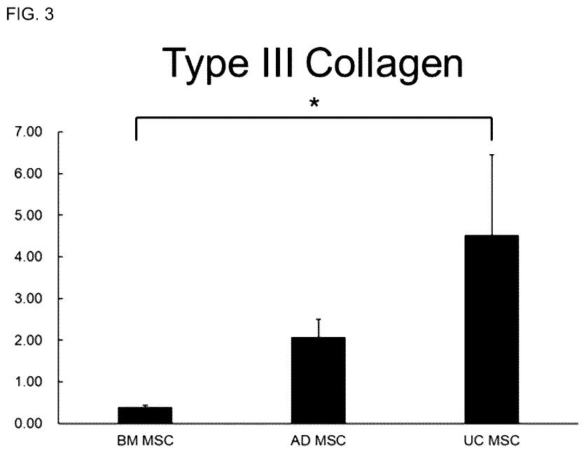

FIGS. 1-3 show a result of quantifying the expression level of the scleraxis, type 1 collagen and type 3 collagen genes in the umbilical cord-derived mesenchymal stem cells (UC MSC) prepared in Example 1, the adipose-derived mesenchymal stem cells (AD MSC) prepared in Comparative Example 1 and the bone marrow-derived stem cells (BM MSC) prepared in Comparative Example 2 by RT-PCR. *P<0.050; **P<0.010

As shown in FIGS. 1-3, the umbilical cord-derived mesenchymal stem cells showed higher mRNA expression of scleraxis, which is a tendon-specific marker, by 1.3 times and 2.3 times as compared to the bone marrow-derived mesenchymal stem cells and the adipose-derived mesenchymal stem cells (P=0.146 and 0.001, respectively).

The umbilical cord-derived mesenchymal stem cells showed higher expression of the type 1 collagen gene by respectively 9.9 times and 2.9 times as compared to the bone marrow-derived mesenchymal stem cells and the adipose-derived mesenchymal stem cells (P=0.009 and 0.027, respectively), and also showed higher mRNA expression of type 3 collagen by 11.6 times and 2.2 times, respectively (P=0.020 and 0.097, respectively).

Accordingly, it was confirmed that the umbilical cord-derived mesenchymal stem cells cultured according to the present disclosure can easily prevent, relieve or treat a tendon disease since it facilitates the regeneration and recovery of tendon cells when applied to tendon injury site.

### Test Example 2. Evaluation of Efficacy Using Rat Rotator Cuff Tear Model

40 male Sprague-Dawley rats (12-week-old, 340-360 g) were divided into 5 groups as descried in Table 1.

Each test group was treated as follows. First, anesthesia was induced using Zoletil and Rompun (30 mg/kg+10 mg/kg). The left shoulder was operated on in all cases. Before initiating surgery, anesthetic depth was checked by slightly applying pressure with a fingernail to the sole of the rat. A 2-cm skin incision was made directly over the anterolateral border of the acromion. After the supraspinatus tendon was exposed by detaching trapezius and deltoid muscles from the acromion, a round full-thickness tear with a diameter of 2 mm (about 50% or larger of the tendon width) was created 1 mm from the supraspinatus tendon and the humeral head using a biopsy punch (BP-20F, Kai Medical Europe GmbH, Bremen, Germany). Then, physiological saline or stem cells were injected adjacent to both sides of the remaining tendon in two divided doses using a 30 G (gauge) needle. After the injection, the trapezius and deltoid muscles were sutured with a 4-0 Vicryl suture (W9074, Ethicon, Cincinnati, OH, USA) and then the skin was also sutured with Black Silk (SK439, AlLee, Busan, Korea) and disinfected. After the surgery, the rats were allowed free cage activity.

The rats of each group were sacrificed at 2 and 4 weeks after the surgery, and the supraspinatus tendon was harvested for macroscopic and histological evaluation.

TABLE 1

| | Experimental design | Remarks |
|---|---|---|
| Control | 10 μL of physiological saline to tendon | 8 |
| group | injury site of full-thickness tendon injury | |
| (saline) | animal model | |
| Test | Umbilical cord-derived mesenchymal stem | 8 |
| group-UC | cells (1 × 10⁶ cells/10 μL of physiological | |
| (UC MSC) | saline) of Example 1 to tendon injury site | |
| | of full-thickness tendon injury animal model | |
| Test | Zkscan8-enhanced umbilical cord-derived | 8 |
| group-ZUC | mesenchymal stem cells (1 × 10⁶ cells/ | |
| (Zkscan8 | 10 μL of physiological saline) of Example | |
| UC MSC) | 2 to tendon injury site of full-thickness | |
| | tendon injury animal model | |
| Comparison | Bone marrow-derived mesenchymal stem | 8 |
| group-BM | cells (1 × 10⁶ cells/10 μL physiological | |
| (BM MSC) | saline) of Comparative Example 2 to tendon | |
| | injury site of full-thickness tendon injury | |
| | animal model | |
| Comparison | Umbilical cord blood-derived mesenchymal | 8 |
| group-UCB | stem cells (1 × 10⁶ cells/10 μL physiological | |
| (UCB MSC) | saline) of Comparative Example 3 to tendon | |
| | injury site of full-thickness tendon injury | |
| | animal model | |

### Test Example 3. Macroscopic Evaluation

At 2 and 4 weeks, the rats of the control group (Saline), the test group-UC (UC-MSC), the comparison group-BM (BM-MSC) and the comparison group-UCB (UCB-MSC) prepared in Test Example 1 were sacrificed in a carbon dioxide chamber, 4 rats per group. The supraspinatus-humerus complex was harvested without removing the humeral head and the supraspinatus muscle to clearly observe the tendon defect.

For macroscopic evaluation of tendon regeneration, a modified semi-quantitative evaluation system of Stoll was used. The 12 parameters in the system were tendon rupture (breakage of tendon), inflammation (swelling, reddening and inflammation of tendon), tendon surface (rough and uneven tendon surface), neighboring tendon (abnormal change in color, thickness and outer surface of neighboring tendon), level of defect (defect site thicker than neighboring tendon), defect size (defect size exceeding 3 mm), swelling/redness of tendon (reddening or swelling of damaged tendon), connection surrounding tissue and slidability (damaged tendon entangled to nearby tissue without slidability), tendon thickness (increased tendon thickness), color of tendon (color of tendon changing from shining white to opaque red), single strain of muscle (supraspinatus tendon connected to neighboring muscle and tissue other than supraspinatus muscle), and transition of construct to surrounding healthy tissue (rough connection of defect site to surrounding healthy tissue, clear distinction of defect). Each parameter varied from 0 or 1 except for swelling/redness of tendon (0 to 2) and tendon thickness (0 to 3). The total macroscopic score varied between 0 (normal tendon) and 15 (most severe injury).

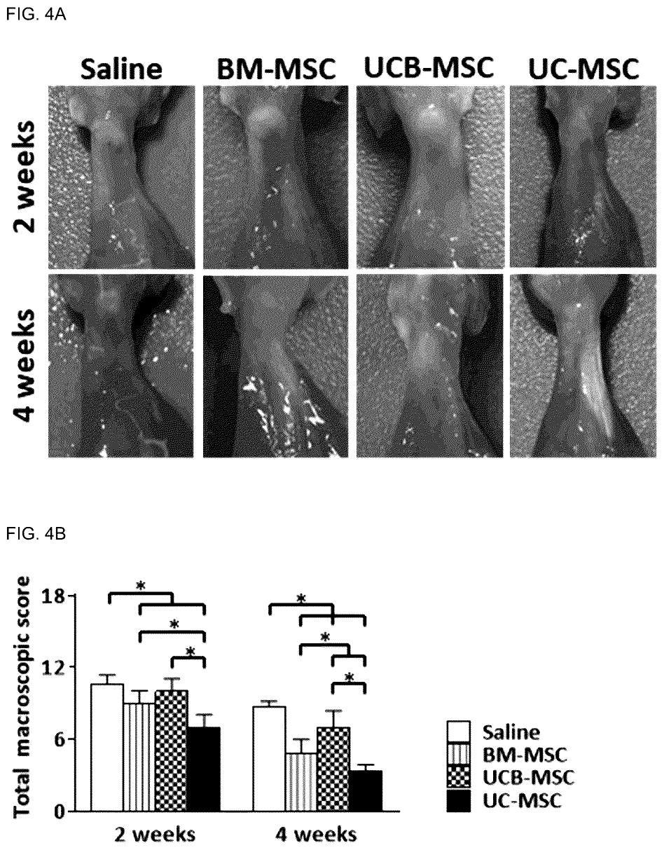

FIGS. 4A and 4B show the images of the supraspinatus tendons of the supraspinatus-humerus complexes obtained at 2 and 4 weeks from control group (Saline), test group-UC (UC-MSC), comparison group-BM (BM-MSC) and comparison group-UCB (UCB-MSC) in Test Example 1.

FIG. 4A shows the macroscopic images of the supraspinatus tendon of each group (tissues around the tendon were removed to clearly observe the defect of the tendon), and FIG. 4B shows the total macroscopic score of the supraspinatus tendon of each group. The graph of FIG. 4B presents mean±standard deviation (SD). *P<0.050.

As shown in FIGS. 4A and 4B, the total macroscopic score at 2 weeks was 7.00±1.07 for the test group-UC (umbilical cord-derived mesenchymal stem cells) and 9.00±1.07 (P=0.002) and 10.00±1.07 (P<0.000), respectively, for the comparison group-BM and the comparison group-UCB. That is to say, when the umbilical cord-derived mesenchymal stem cells were administered, the tendon injury was recovered remarkably and significantly within a short period as compared to other stem cells.

Especially, the test group-UC showed lower scores in neighboring tendon, level of defect and swelling/redness of tendon by at least 0.5 point.

At 4 weeks, the total macroscopic score was 3.38±7.00 for the test group-UC (umbilical cord-derived mesenchymal stem cells), 4.88±1.13 (P=0.012) for the comparison group-BM and 7.00±1.41 (P<0.000) for the comparison group-UCB. That is to say, when the umbilical cord-derived mesenchymal stem cells were administered, the tendon injury was recovered significantly as compared to other stem cells.

Especially, the test group-UC showed lower scores in swelling/redness of tendon, connection surrounding tissue and slidability, and tendon thickness by at least 0.5 point.

There is a concern that, when umbilical cord-derived mesenchymal stem cells are used as a cell therapy agent for treating a tendon disease, immune response may be induced against heteroplastic transplantation and allogeneic transplantation since they function as a target for innate and acquired immune response as NK, T- and B-cells.

However, the umbilical cord-derived mesenchymal stem cells of Example 1 showed no special rejection even after heteroplastic transplantation into the rat. On the contrary, the umbilical cord-derived stem cells showed significantly lower damage in terms of neighboring tendon, level of defect and swelling/redness of tendon as compared to other stem cells.

It is thought that the umbilical cord-derived mesenchymal stem cells according to the present disclosure can be heteroplastically transplantated effectively for tendon injury because they control immune response by regulating macrophages and T-lymphocytes, reduce natural cell death and are favorable for engraftment as compared to other stem cells.

In addition, there is a problem that, because damaged tendon is recovered as it is attached to nearby tissue, restricted motion or pain occurs after the recovery. The umbilical cord-derived mesenchymal stem cells according to the present disclosure showed connection surrounding tissue and slidability lower by at least 0.5 point and transition of construct to surrounding healthy tissue, single strain of muscle, etc. improved by at least 0.5 point as compared to other stem cells, indicating that the umbilical cord-derived mesenchymal stem cells are the most desirable for prevention, relieving and treatment of tendon diseases.

### Test Example 4. Evaluation of Degenerative Change and Integration of Structure of Tendon

The rats of the control group (Saline), the test group-UC (UC-MSC), the comparison group-BM (BM-MSC) and the comparison group-UCB (UCB-MSC) prepared in Test Example 1 were sacrificed in a carbon dioxide chamber, 4 rats per group, at 2 and 4 weeks. The supraspinatus-humerus complex was harvested without removing the humeral head and the supraspinatus muscle to clearly observe the tendon defect.

After isolating tendon tissues from the harvested supraspinatus-humerus complex of each group, the tendon tissues were immediately fixed in 4% (w/v) paraformaldehyde (PFA; Merck, Germany) for 24 hours and decalcified in 10% ethylendiaminetetracetic acid (EDTA; Sigma-Aldrich, St Louis, MO, USA) for two days. Subsequently, the tissues were dehydrated in an increasing ethanol gradient and defatted in chloroform. The fixed tendon tissues were embedded in paraffin blocks and trimmed carefully to the middle part of the tendon and then cut into 4 mm-thick serial slides using a microtome.

After randomly selecting a slide from each group, it was stained with hematoxylin and eosin (H&E) and an image was obtained using an optical microscope (U-TVO 63XC; Olympus Corp., Japan).

The tendinopathy of each group was evaluated using the image. Each slide was evaluated using the modified semi-quantitative evaluation method of Astrom and Movin (Jo C H, Shin W H, Park J W, Shin J S, Kim J E. Degree of tendon degeneration and stage of rotator cuff disease. Knee Surg Sport Tr A. 2017; 25(7): 2100-8). The 7 parameters of the system include fiber structure (finely split long collagens), fiber arrangement (change in collagen fiber arrangement from parallel to irregular), rounding of nuclei (rounding of the flat nuclei of fibroblasts due to damage or activation), variations in cellularity (increased number and clustering of cells in the tendon), increased vascularity (increased number and size of blood vessels in the tendon), decreased stainability (decreased stainability due to decreased fiber density caused by damage to fibers), and hyalinization (change of collagen fibers in tendon tissues to hyaline material). The total degeneration score varies between 0 (normal tendon) and 21 (most severely degenerated).

Next, the integration of structure was evaluated from the optical image of the slide of each group. The integration of structure is for evaluating the connection between the defect site and an intact site. The integration of structure was evaluated according to the Burgisser's method from 0 to 3 points; 0 (no gap), 1 (recognizable change), 2 (abrupt change, recognizable gap or callus tissue) and 3 (void defect site) (Meier Burgisser G, Calcagni M, Bachmann E, Fessel G, Snedeker J G, Giovanoli P, et al. Rabbit Achilles tendon full transection model—wound healing, adhesion formation and biomechanics at 3, 6 and 12 weeks post-surgery. Biol Open. 2016; 5(9): 1324-33).

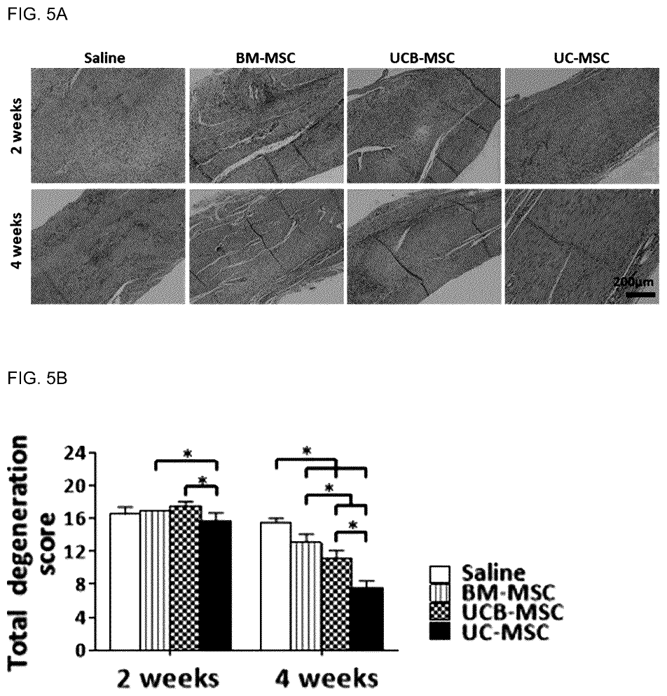

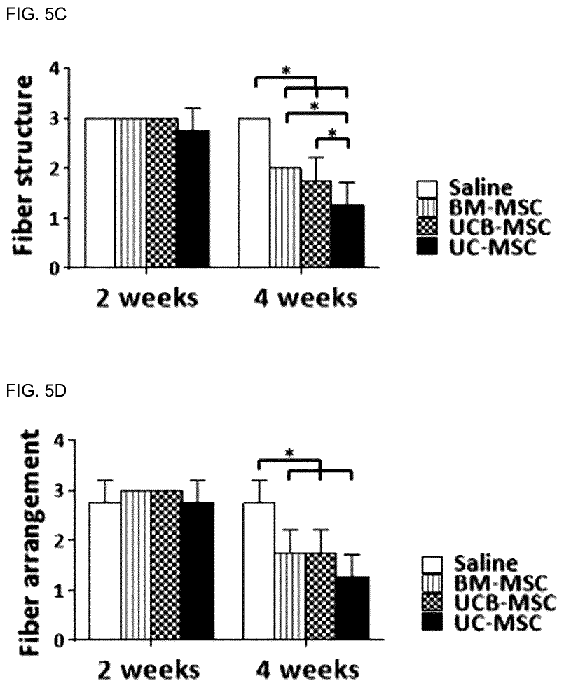

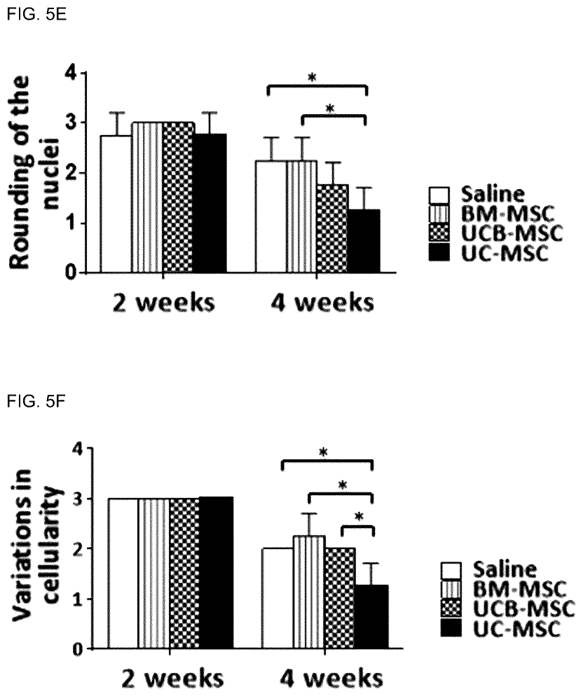

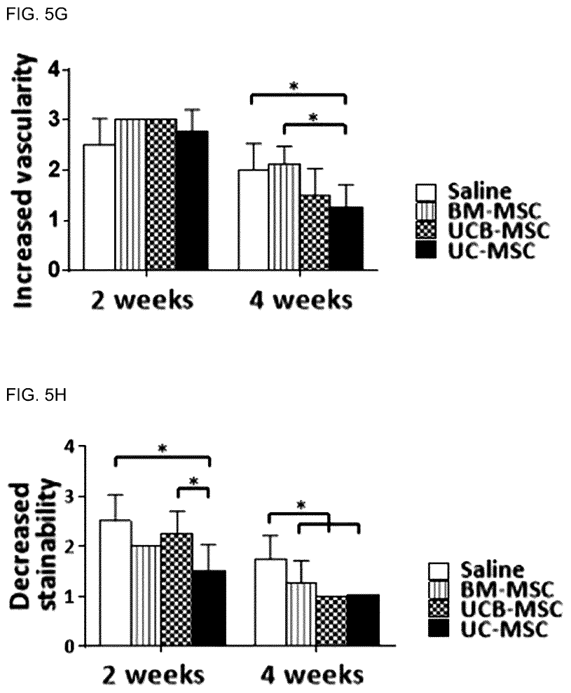

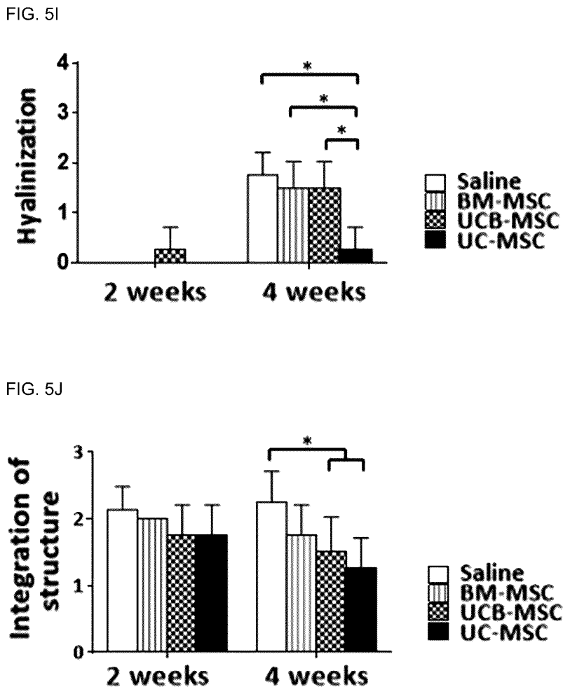

FIG. 5 shows a result of recovering tendon tissues from the supraspinatus-humerus complexes obtained at 2 and 4 weeks from control group (Saline), test group-UC (UC-MSC), comparison group-BM (BM-MSC) and comparison group-UCB (UCB-MSC) in Test Example 1 and evaluating degenerative change and integration of structure. FIG. 5A shows the optical microscopic images of the tendons obtained at 2 and 4 weeks from control group (Saline), test group-UC (UC-MSC), comparison group-BM (BM-MSC) and comparison group-UCB (UCB-MSC) in Test Example 1 and stained with H&E (magnification: ×200). FIG. 5B shows the total degeneration scores of the tendons obtained at 2 and 4 weeks from control group (Saline), test group-UC (UC-MSC), comparison group-BM (BM-MSC) and comparison group-UCB (UCB-MSC) in Test Example 1, FIG. 5C-5I show the sub-parameters of the degeneration scores, and FIG. 5J shows the integration of structure. Each graph presents mean±standard deviation (SD). *P<0.050.

As shown in FIG. 5, the total degeneration score at 2 weeks was 15.50±1.20 for the umbilical cord-derived mesenchymal stem cells (test group-UC), 17.00±0.00 (P=0.005) for the comparison group-BM and 17.50±0.53 (P<0.001) for the comparison group-UCB. That is to say, when the umbilical cord-derived mesenchymal stem cells were administered, the degenerative change of the tendon tissue was significantly decreased within a short period of time as compared to other stem cells. Through this, it can be seen that the umbilical cord-derived mesenchymal stem cells have significantly superior effect of recovering tendon injury as compared to the umbilical cord blood-derived mesenchymal stem cells.

At 4 weeks, the total degeneration score was 7.50±0.93 for the umbilical cord-derived mesenchymal stem cells (test group-UC), 13.13±0.99 (P<0.001) for the comparison group-BM and 11.25±0.89 (P=0.050) for the comparison group-UCB. That is to say, the administration of the umbilical cord-derived mesenchymal stem cells resulted in significantly decreased tendinopathy as compared to other stem cells. Especially, the umbilical cord-derived mesenchymal stem cells showed significant recovery in terms of fiber structure, rounding of nuclei, variations in cellularity, increased vascularity and hyalinization as compared to other stem cells.

As shown in FIG. 5J, the integration of structure indicating the connection of the defect site to the intact site was 1.25±0.46 for the umbilical cord-derived mesenchymal stem cells (test group-UC), 2.25±0.46 (P=0.002) for the control group, 1.75±0.46 for the comparison group-BM and 1.50±0.53 for the comparison group-UCB. That is to say, whereas the bone marrow-derived mesenchymal stem cells and the umbilical cord blood-derived mesenchymal stem cells did not show significant recovery as compared to the control group, the umbilical cord-derived mesenchymal stem cells of the present disclosure showed significant recovery as compared to the control group.

### Test Example 5. Evaluation of Collagen Tissue and Fibroblasts of Tendon

The slide of each group prepared in Test Example 4 was stained with picrosirius red (PSR) and an image was obtained using a circularly polarized optical microscope. Collagen organization (production and organization of collagen) and collage fiber coherence (coherent arrangement of collagen fibers of the tendon) were analyzed using the image. The collagen organization was measured as intense white areas of brightly diffracted light on gray scale (black, 0; white, 255) using the ImageJ software. Higher gray scale indicates the formation of more organized and mature collagen (Zhao S, Zhao J W, Dong S K, Huangfu X Q, Bin L, Yang H L, et al. Biological augmentation of rotator cuff repair using bFGF-loaded electrospun poly(lactide-co-glycolide) fibrous membranes. Int J Nanomed. 2014; 9: 2373-85).

The collage fiber coherence is a measure of the extent of collagen fiber alignment in the major axis of the tendon. The coherence was quantified using the program called Orientation J plug-in for ImageJ. Five regions were analyzed for the slide of each group and the mean value was multiplied by 100 to obtain the final coherence value (Degen R M, Carbone A, Carballo C, Zong J C, Chen T, Lebaschi A, et al. The Effect of Purified Human Bone Marrow-Derived Mesenchymal Stem Cells on Rotator Cuff Tendon Healing in an Athymic Rat. Arthroscopy. 2016; 32(12): 2435-43).

Then, fibroblasts were evaluated. In normal tendon, the few fibroblasts with flattened nuclei are aligned parallel to the tensile axis. In damaged tendon, the number of fibroblasts is increased and, at the same time, the nuclei become round and the cells are skewed in different directions. For the tendon tissue of each group, fibroblast density (fibroblast density is increased as the damage is severe), nuclear aspect ratio (the nuclei of the cells become round when the cells are damaged) and nuclear orientation angle (nuclear orientation angle is increased as nearby tissues or fibroblasts are damaged) were evaluated. Five regions were measured for the slide of each group and the average was recorded.

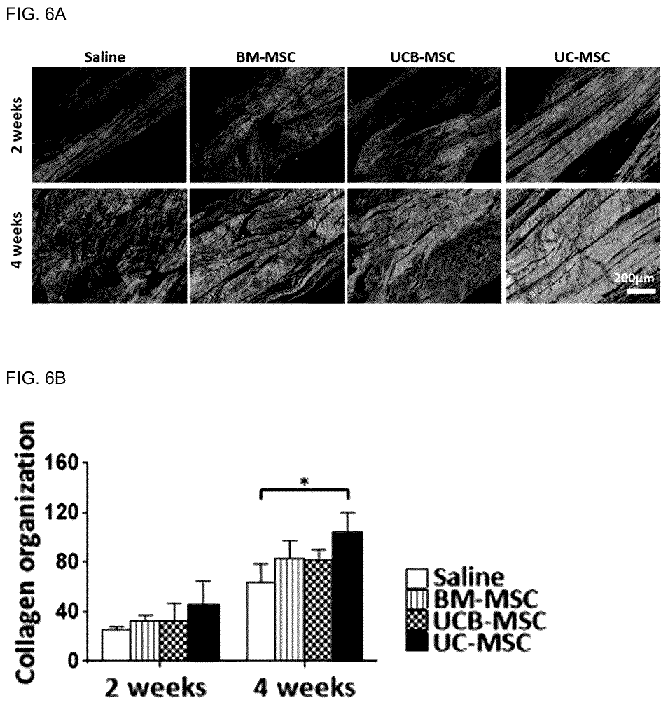

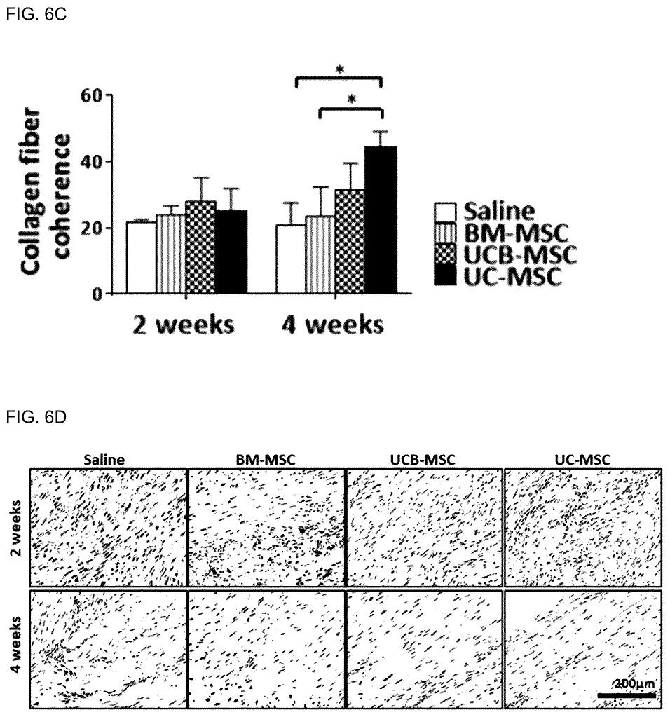

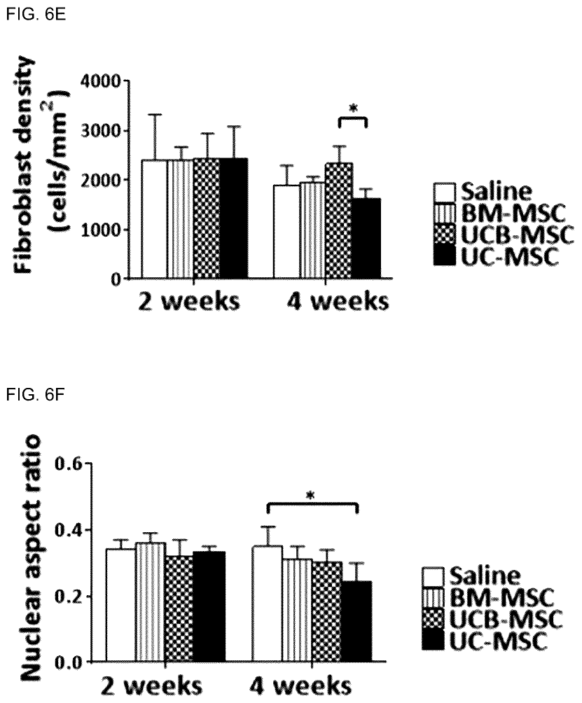

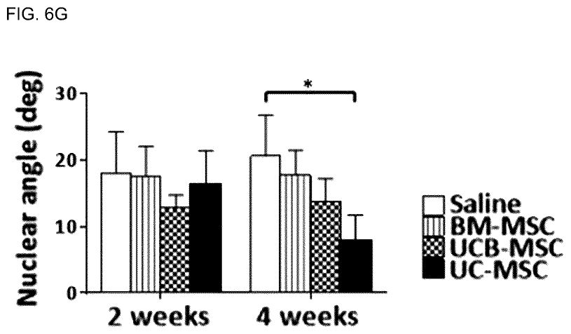

FIG. 6 shows a result of evaluating collagen tissues and fibroblasts in tendon tissues of the supraspinatus-humerus complexes obtained at 2 and 4 weeks from control group (Saline), test group-UC (UC-MSC), comparison group-BM (BM-MSC) and comparison group-UCB (UCB-MSC) in Test Example 1. FIG. 6A shows the optical microscopic images of the tendons obtained at 2 and 4 weeks from control group (Saline), test group-UC (UC-MSC), comparison group-BM (BM-MSC) and comparison group-UCB (UCB-MSC) in Test Example 1 and stained with PSR (magnification: ×200). FIG. 6B shows a result of evaluating the collagen organization of the tendons obtained at 2 and 4 weeks from control group (Saline), test group-UC (UC-MSC), comparison group-BM (BM-MSC) and comparison group-UCB (UCB-MSC) in Test Example 1, and FIG. 6C shows a result of evaluating collage fiber coherence. FIG. 6D shows the images of fibroblasts in the tendons obtained at 2 and 4 weeks from control group (Saline), test group-UC (UC-MSC), comparison group-BM (BM-MSC) and comparison group-UCB (UCB-MSC) in Test Example 1 (magnification: ×400), FIG. 6E shows a result of evaluating fibroblast density, FIG. 6F shows a result of evaluating the nuclear aspect ratio of the fibroblasts, and FIG. 6G shows a result of evaluating nuclear orientation angle. The graphs present mean±standard deviation (SD). *P<0.050.

As shown in FIG. 6, the collagen organization score at 4 weeks was 103.60±16.88 for the umbilical cord-derived mesenchymal stem cells (test group-UC). The collagen organization score for the control group, the comparison group-BM and the comparison group-UCB was 63.36±15.45 (P=0.002), 83.30±14.30 and 82.19±8.21, respectively. That is to say, whereas the bone marrow-derived mesenchymal stem cells and the umbilical cord blood-derived mesenchymal stem cells did not show significant recovery as compared to the control group, the umbilical cord-derived mesenchymal stem cells showed significant recovery as compared to the control group.

The collage fiber coherence (FIG. 6C) was 44.15±4.94 for the umbilical cord-derived mesenchymal stem cells (test group-UC), 20.88±6.80 (P=0.002) for the control group, 23.61±8.86 (P=0.006) for the comparison group-BM and 31.29±8.21 for the comparison group-UCB. That is to say, whereas the collage fiber coherence was hardly recovered for the bone marrow-derived mesenchymal stem cells and the umbilical cord blood-derived mesenchymal stem cells as compared to the control group, the umbilical cord-derived mesenchymal stem cells showed significant recovery of the collage fiber coherence as compared to the control group.

The fibroblast density score (FIG. 6D-6G) was 1594.93±221.90 cells/mm² for the umbilical cord-derived mesenchymal stem cells (test group-UC), 1887.71±407.93 cells/mm² for the control group, 1944.60±117.16 cells/mm² for the comparison group-BM and 2335.03±350.40 cells/mm² for the comparison group-UCB. That is to say, whereas the bone marrow-derived mesenchymal stem cells and the umbilical cord blood-derived mesenchymal stem cells showed little change in fibroblast density as compared to the control group, the umbilical cord-derived mesenchymal stem cells showed significant decrease in the fibroblast density score as compared to the control group.

The nuclear aspect ratio of fibroblasts (FIG. 6F) was 0.24±0.06 for the umbilical cord-derived mesenchymal stem cells (test group-UC), 0.35±0.06 for the control group, 0.31±0.04 for the comparison group-BM and 0.30±0.04 for the comparison group-UCB. That is to say, whereas the nuclear aspect ratio of fibroblasts of the bone marrow-derived mesenchymal stem cells and the umbilical cord blood-derived mesenchymal stem cells was similar to that of the control group, the nuclear aspect ratio of fibroblasts of the umbilical cord-derived mesenchymal stem cells was significantly decreased as compared to the control group.

The nuclear orientation angle of fibroblasts (FIG. 6G) was 7.75±4.01 for the umbilical cord-derived mesenchymal stem cells (test group-UC), 18.05±6.20 for the control group, 17.73±3.75 for the comparison group-BM and 13.76±3.47 for the comparison group-UCB. That is to say, whereas the nuclear orientation angle of fibroblasts of the bone marrow-derived mesenchymal stem cells and the umbilical cord blood-derived mesenchymal stem cells was similar to that of the control group, the nuclear orientation angle of fibroblasts of the umbilical cord-derived mesenchymal stem cells was decreased remarkably as compared to the control group.

### Test Example 6. Evaluation of Heterotopic Change in Tendon

After staining the slide of each group prepared in Test Example 4 with safranin O-fast green (Saf-O), an image was obtained using an optical microscope. The glycosaminoglycan-rich area (stained red) was analyzed from the image (entire tendon tissue) using the ImageJ software. The glycosaminoglycan (GAG)-rich area (nonspecific formation of cartilage tissue in tendon tissue) can be used as a measure of heterotopic cartilage formation because.

In addition, for evaluation of heterotopic ossification (nonspecific formation of bone tissue in tendon tissue), the slide of each group prepared in Test Example 4 was stained with H&E and then the areas of separation, clustering and bar-shaped foci were analyzed using ImageJ.

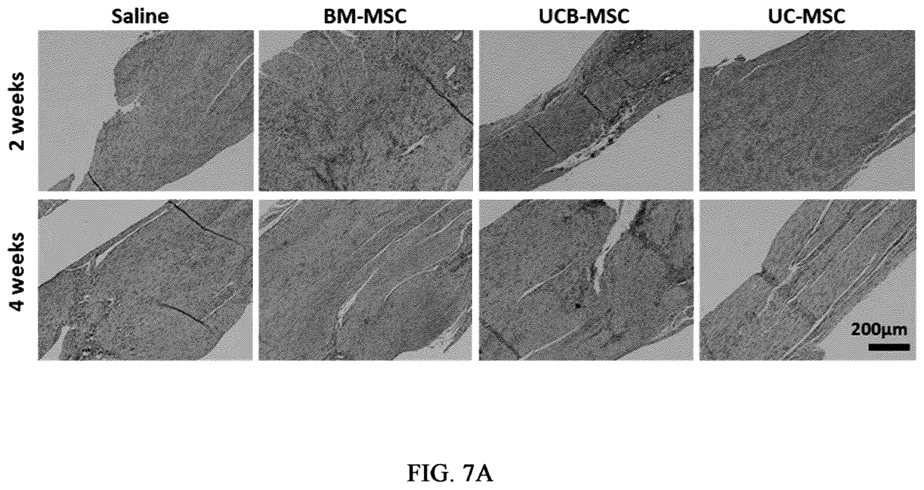

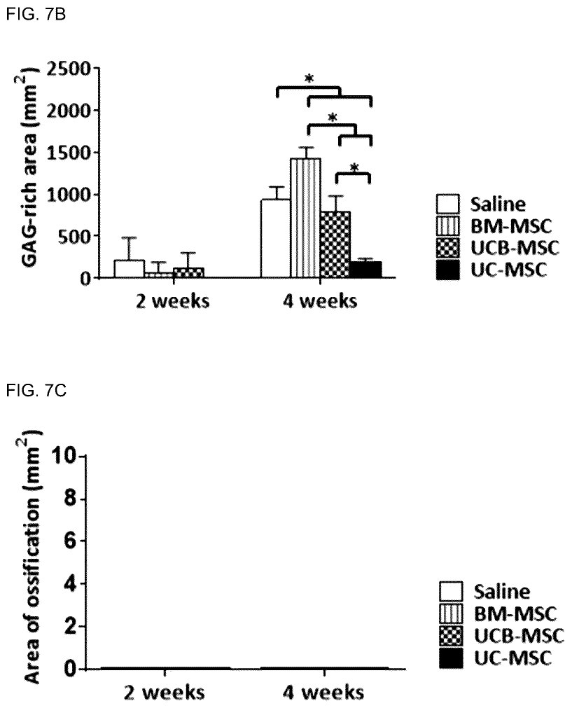

FIG. 7 shows a result of analyzing heterotopic change in the tendon tissues of the supraspinatus-humerus complexes obtained at 2 and 4 weeks from control group (Saline), test group-UC (UC-MSC), comparison group-BM (BM-MSC) and comparison group-UCB (UCB-MSC) in Test Example 1. FIG. 7A shows the images of the tendons obtained at 2 and 4 weeks from control group (Saline), test group-UC (UC-MSC), comparison group-BM (BM-MSC) and comparison group-UCB (UCB-MSC) in Test Example 1 and stained with Saf-O (magnification: ×200). FIG. 7B shows a result of measuring the glycosaminoglycan (GAG)-rich area of the tendons obtained at 2 and 4 weeks from control group (Saline), test group-UC (UC-MSC), comparison group-BM (BM-MSC) and comparison group-UCB (UCB-MSC) in Test Example 1, and FIG. 7C shows a result of measuring the area of ossification of the tendons obtained at 2 and 4 weeks from control group (Saline), test group-UC (UC-MSC), comparison group-BM (BM-MSC) and comparison group-UCB (UCB-MSC) in Test Example 1. The graphs present mean±standard deviation (SD). *P<0.050.

As shown in FIG. 7, the glycosaminoglycan-rich area at 4 weeks was 176.16±63.28 mm² for the umbilical cord-derived mesenchymal stem cells (test group-UC), 939.50±148.66 mm² (P<0.000) for the control group, 1428.32±134.16 mm² (P<0.000) for the comparison group-BM and 788.64±194.95 mm² (P<0.000) for the comparison group-UCB. That is to say, whereas heterotopic cartilage formation was increased for the bone marrow-derived mesenchymal stem cells as compared to the control group and similar for the umbilical cord blood-derived mesenchymal stem cells to the control group, the heterotopic cartilage formation was significantly decreased for the umbilical cord-derived mesenchymal stem cells as compared to the control group (FIG. 7B).

Heterotopic ossification was not observed in any groups, including the umbilical cord-derived mesenchymal stem cells.

The main cause of unsuccessful tendon healing is the formation of a scar tissue consisting of disorganized collagen fibers during the recovery of the damaged tendon. Through the experiments described above, it was confirmed that the umbilical cord-derived mesenchymal stem cells of the present disclosure can fill the defect site of tendon with normal tendon tissue, instead of scar tissue, by significantly helping collagen production, organization and alignment as compared to the bone marrow-derived mesenchymal stem cells or the umbilical cord blood-derived mesenchymal stem cells.

In addition, whereas stem cells may exhibit side effects such as shoulder pain, retear and complications due to heterotopic cartilage formation and ossification, the umbilical cord-derived mesenchymal stem cells of the present disclosure inhibited heterotopic cartilage formation and did not induce heterotopic ossification unlike the bone marrow-derived mesenchymal stem cells or the umbilical cord blood-derived mesenchymal stem cells. Therefore, it can be seen that they can recover the normal function of tendon tissue without side effects when clinically applied to tendon diseases.

Accordingly, it was confirmed that, when applied to a tendon disease, especially full-thickness tendon injury of the rotator cuff, the umbilical cord-derived mesenchymal stem cells are significantly effective in macroscopic and histological aspects as compared to other stem cells such as the bone marrow-derived mesenchymal stem cells and the umbilical cord blood-derived mesenchymal stem cells, and can recover the normal function of tendon tissue without side effects such as heterotopic ossification.

### Test Example 7. Macroscopic Evaluation of Zkscan8 Gene-Transduced Umbilical Cord-Derived Mesenchymal Stem Cells

The experiments were approved by the Institutional Animal Care and Use Committee and conducted according to approved procedures (IACUC_2019_0044). 40 male Sprague-Dawley rats (12-week-old, 340-360 g) were divided into 4 groups: (1) normal group (Normal); 2) physiological saline group (Saline); 3) umbilical cord-derived mesenchymal stem cell group (MSC); and 4) Zkscan8 gene-transduced umbilical cord-derived mesenchymal stem cell group (MSC-Zk8)).

Anesthesia was induced using Zoletil (30 mg/kg) and Rompun (10 mg/kg). The left shoulder was operated on in all cases. Before initiating surgery, anesthetic depth was checked by slightly applying pressure with a fingernail to the sole of the rat. A 2-cm skin incision was made directly over the anterolateral border of the acromion. After the supraspinatus tendon was exposed by detaching trapezius and deltoid muscles from the acromion, a round full-thickness tear with a diameter of 2 mm (about 50% or larger of the tendon width) was created 1 mm from the supraspinatus tendon and the humeral head using a biopsy punch (BP-20F, Kai Medical Europe GmbH, Bremen, Germany). Then, 2) 10 μL of physiological saline, 3) umbilical cord-derived mesenchymal stem cells (1×10⁶ cells/10 μL physiological saline) or 4) Zkscan8 gene-transduced umbilical cord-derived mesenchymal stem cells (1×10⁶ cells/10 μL physiological saline) were injected adjacent to both sides of the remaining tendon in two divided doses using a 30 G (gauge) needle. After the injection, the trapezius and deltoid muscles were sutured with a 4-0 Vicryl suture (W9074, Ethicon, Cincinnati, OH, USA) and then the skin was also sutured with Black Silk (SK439, AILee, Busan, Korea) and disinfected. After the surgery, the rats were allowed free cage activity.

The rats of each group were sacrificed at 2 and 4 weeks after the surgery, and the supraspinatus tendon was harvested and used for macroscopic, histological and biomechanical evaluation.

At 2 and 4 weeks after the surgery, the rat of each group was sacrificed in a carbon dioxide chamber. The supraspinatus tendon of the rat was harvested without removing the humeral head and the supraspinatus muscle. The modified semi-quantitative system of Stoll described in Test Example 3 was used for macroscopic evaluation of tendon regeneration (Stoll C, John T, Conrad C et al. Healing parameters in a rabbit partial tendon defect following tenocyte/biomaterial implantation. Biomaterials 2011; 32(21): 4806-4815).

FIG. 11 shows the macroscopic images of the supraspinatus tendons of the normal group (Normal), physiological saline group (Saline), umbilical cord-derived mesenchymal stem cell group (MSC) and Zkscan8 gene-transduced umbilical cord-derived mesenchymal stem cell group (MSC-Zk8) at 2 and 4 weeks. The tissue around the defect site was removed for clear observation of the tendon defect.

FIG. 12 shows a result of analyzing the total macroscopic score for the supraspinatus tendons of the normal group (Normal), physiological saline group (Saline), umbilical cord-derived mesenchymal stem cell group (MSC) and Zkscan8 gene-transduced umbilical cord-derived mesenchymal stem cell group (MSC-Zk8) at 2 and 4 weeks. The graphs present mean±standard deviation (SD). *P<0.050.

The total macroscopic score, which is a measure of severe apparent damage, of each group is compared in FIG. 11 and FIG. 12. At 2 weeks, the total macroscopic score was 4.75±0.46 for the Zkscan8 gene-transduced umbilical cord-derived mesenchymal stem cell group (Example 2) (MSC-Zk8). But, the physiological saline group and the umbilical cord-derived mesenchymal stem cell group showed severe damage with 10.75±1.28 (P<0.000) and 7.25±0.89 (P<0.000), respectively. In particular, the Zkscan8 gene-transduced umbilical cord-derived mesenchymal stem cell group (MSC-Zk8) showed lower scores (less damage) in inflammation, connection surrounding tissue and slidability, and tendon thickness as compared to other groups.

At 4 weeks, the total macroscopic score was 2.75±0.46 for the Zkscan8 gene-transduced umbilical cord-derived mesenchymal stem cell group (MSC-Zk8), 9.00±0.00 (P<0.000) for the physiological saline group and 4.25±0.89 (P<0.000) for the umbilical cord-derived mesenchymal stem cell group. At 4 weeks, the Zkscan8 gene-transduced umbilical cord-derived mesenchymal stem cell group showed a lower score than the umbilical cord-derived mesenchymal stem cell group.

From the above experiments, it can be seen that the overexpression of Zkscan8 can improve the tissue damage-recovering ability of the umbilical cord-derived mesenchymal stem cells. It was confirmed that the Zkscan8 gene-transduced umbilical cord-derived mesenchymal stem cells (MSC-Zk8) exhibit an effect of preventing or treating the pain and symptoms of patients that can be caused by tendon injury in short time as compared to the umbilical cord-derived mesenchymal stem cells.

1-6. (canceled)

7. A method for treating a tendon or ligament disease in a subject in need thereof, comprising administering to the subject umbilical cord-derived mesenchymal stem cells.

8. The method of claim 7, wherein the umbilical cord-derived stem cells express the scleraxis gene, the type 1 collagen gene, and the type 3 collagen gene.

9. The method of claim 7, wherein the umbilical cord-derived mesenchymal stem cells are transduced with a nucleic acid sequence encoding Zkscan8(zinc finger protein with KRAB and SCAN domains 8).

10. The method of claim 9, wherein the Zkscan8 comprises the amino acid sequence of SEQ ID NO: 2

11. The method of claim 7, wherein
the tendon disease is one or more selected from a group consisting of Achilles tendon disease, patellar tendon disease, lateral epicondylitis, medial epicondylitis, plantar fasciitis, rotator cuff tendon disease, tenosynovitis, tendinopathy, tendinitis, tenosynovitis, tendon injury and tendon avulsion, and
the ligament disease is one or more selected from a group consisting of cruciate ligament injury, ankle ligament injury, collateral ligament injury, ligament rupture and ligament sprain.

12. The method of claim 7, wherein the umbilical cord-derived mesenchymal stem cells prevent or treat heterotopic ossification induced during the regeneration of damaged tendon or ligament.

13. A method for treating a heterotopic ossification caused by a tendon or ligament disease in a subject in need thereof, comprising administering to the subject umbilical cord-derived mesenchymal stem cells.

14. The method of claim 13, wherein the heterotopic ossification is induced during the regeneration of damaged tendon or ligament.

15. The method of claim 13, wherein the umbilical cord-derived mesenchymal stem cells are transduced with a nucleic acid sequence encoding Zkscan8 (zinc finger protein with KRAB and SCAN domains 8),

16. The method of claim 14, wherein the Zkscan8 comprises the amino acid sequence of SEQ ID NO: 2.


SEQUENCE LISTING (5 sequences)

SEQ ID NO: 1 - DNA, 1737 bp, Artificial Sequence
  Zkscan 8(Zinc finger protein with KRAB and SCAN domains 8)
atggctgaagaatcaagaaagccttcagccccatccccaccagaccagactcctgaagaggatcttgtaatcgtcaaggtagaggaggatcatggttgggaccaggaatctagtctgcatgaaagtaaccctcttggccaagaagtgttccgcctgcgcttcaggcagttacgctaccaggagacactaggaccccgagaagctctgatccaactacgggccctttgccatcagtggctgaggccagatttgaacaccaaggaacagatcctggagctgctggtgctggagcagttcttgaccatcctacctgaggagctccagacactggttaaggaacatcagctagagaacggagaggaggtggtgaccctattagaggatttggaaaggcagattgatatactaggacgaccagtctcagctcgcgtacatggacatagggtactctgggaggaggtagtacattcagcatctgcaccagagcctccaaatactcagctccaatctgaggcaacccaacataaatctccagtgccccaagagtcacaagagagagccatgtctacttcccagagtcctactcgttcccagaaaggaagttctggagaccaggaaatgacagctacacttctcacagcagggttccagactttggagaagattgaagacatggctgtgtcccttattcgagaggagtggcttcttgatccatcacagaaggatctgtgtagagataacaggccagaaaatttcagaaacatgttctccctgggtggtgagaccaggagtgagaacagggaattagcttcaaaacaggtaatatctactggaatccagccacatggagagacagctgccaaatgcaacggggatgttatcaggggtcttgagcatgaagaagcccgagaccttctgggcagattagagaggcagcggggaaatcccacacaagagagacgacataaatgtgatgaatgtgggaaaagctttgctcagagctcaggccttgttcgccactggagaatccacactggggagaaaccctatcagtgtaatgtgtgtggtaaagccttcagttacaggtcagcccttctttcacatcaggatatccacaacaaagtaaaacgctatcactgtaaggagtgtggcaaagccttcagtcagaacacaggcctgattctgcaccagagaatccacactggggagaagccatatcagtgcaatcagtgtgggaaggctttcagtcagagtgcgggccttattctgcaccagagaatccacagtggagagagaccctatgaatgtaatgagtgtgggaaagctttcagtcatagctcacacctcattggacatcagagaatccacactggggagaagccctatgagtgtgatgagtgtgggaaaaccttcaggcggagctcacatcttattggtcatcagaggagccacactggggagaaaccctacaaatgcaatgagtgtgggagggccttcagtcagaagtcaggccttattgaacatcagagaatccacactggagaaagaccctataaatgtaaagaatgtgggaaagctttcaatgggaacactggtctcattcaacacctgagaattcacacaggggagaagccctaccaatgtaatgagtgtgggaaagcctttattcagaggtcaagtctcattcgacatcagagaatccacagtggtgaaaaatctgaatccataagcgtttag

SEQ ID NO: 2 - protein, 578 aa, Artificial Sequence
  Zkscan 8(Zinc finger protein with KRAB and SCAN domains 8)
MAEESRKPSAPSPPDQTPEEDLVIVKVEEDHGWDQESSLHESNPLGQEVFRLRFRQLRYQETLGPREALIQLRALCHQWLRPDLNTKEQILELLVLEQFLTILPEELQTLVKEHQLENGEEVVTLLEDLERQIDILGRPVSARVHGHRVLWEEVVHSASAPEPPNTQLQSEATQHKSPVPQESQERAMSTSQSPTRSQKGSSGDQEMTATLLTAGFQTLEKIEDMAVSLIREEWLLDPSQKDLCRDNRPENFRNMFSLGGETRSENRELASKQVISTGIQPHGETAAKCNGDVIRGLEHEEARDLLGRLERQRGNPTQERRHKCDECGKSFAQSSGLVRHWRIHTGEKPYQCNVCGKAFSYRSALLSHQDIHNKVKRYHCKECGKAFSQNTGLILHQRIHTGEKPYQCNQCGKAFSQSAGLILHQRIHSGERPYECNECGKAFSHSSHLIGHQRIHTGEKPYECDECGKTFRRSSHLIGHQRSHTGEKPYKCNECGRAFSQKSGLIEHQRIHTGERPYKCKECGKAFNGNTGLIQHLRIHTGEKPYQCNECGKAFIQRSSLIRHQRIHSGEKSESISV

SEQ ID NO: 3 - DNA, 1737 bp, Artificial Sequence
  Optimized zkscan8 gene sequence
atggcggaggaaagtcggaaaccatccgcccccagtccgcccgatcaaacccctgaagaagaccttgtcatagtcaaagtcgaggaggaccacggctgggatcaggaaagcagccttcacgagtctaacccgcttggacaagaggtttttcgcctgagatttcgtcagttgcgttaccaggagactcttggtccacgggaagctcttatacaactgagagcattgtgtcaccaatggttgcggccagaccttaatactaaagagcagatattagaactgctggttttggagcaatttttgacaatactgccggaggaactgcaaactcttgtcaaagagcaccaactggaaaatggtgaggaagttgtaacccttcttgaggacctggagagacagattgatatccttggccgcccagtatcagcgcgcgtgcatggccatcgcgtcttgtgggaagaggtcgtccatagcgcaagtgcccctgagccaccaaacactcagcttcagagcgaggcgacacagcataagtcgcctgttcctcaggagagccaggaacgggccatgagtaccagtcagtcgcctacaagaagccaaaagggctctagtggggatcaggaaatgacagccacgttacttaccgcagggttccagaccttagaaaaaatcgaggatatggccgtatcgttgatccgggaagagtggctgcttgacccatctcagaaggatctgtgcagagacaatcggcctgagaacttccgcaatatgttctcccttggtggagaaacgcggtcggaaaatagagagctggcaagcaagcaagttatcagcacaggcatccagcctcatggcgaaacggctgctaaatgtaatggtgatgtcatacgtgggttagaacatgaggaagcccgggatttattggggcgtctggaacgccagcgtggaaatccgacacaggagcggcgtcataaatgcgatgaatgtggtaagagtttcgctcaatcgtctgggcttgttcggcattggcggattcatacgggagagaagccttaccagtgtaatgtatgcgggaaagcgttttcataccggtctgcccttctttcacatcaagatatccataacaaggtcaaacgttatcactgcaaagaatgcggtaaagcgttttctcagaatacggggctgatcttacatcagagaatccataccggagaaaaaccttatcaatgcaatcagtgcggaaaggccttttctcagagcgcagggctgattttgcatcaaagaattcattcaggcgagcgtccctatgagtgtaatgagtgcggcaaagcttttagccacagctcgcacctgattggtcatcaacgcattcacacgggtgaaaagccctatgaatgcgatgagtgcggaaagacgtttcgccgctcgtcgcatttaataggccaccaacgttctcatacaggagaaaaaccgtacaagtgcaatgaatgtgggagagccttcagccagaaaagcggtctgatagagcaccaacgtatccacacgggggagagaccgtacaaatgcaaagaatgtgggaaagcctttaatggaaataccgggctgattcagcacctgagaattcacactggagagaagccttaccaatgtaacgagtgcggtaaagcttttatacaacgttcgagtttaatacgccatcaacggatacacagtggtgagaaatcagagtctatctcagtctaa

SEQ ID NO: 4 - DNA, 56 bp, Artificial Sequence
  Primer
aaggatccatgtacccatacgatgttccagattacgctatggcggaggaaagtcgg

SEQ ID NO: 5 - DNA, 26 bp, Artificial Sequence
  Primer
aagtcgacctagactgagatagactc